(19) United States
(12) Patent Application Publication
(10) Pub. No.: US 2023/0139028 A1
(43) Pub. Date: May 4, 2023

(54) METHOD AND ARRANGEMENT FOR TESTING A HUMAN PHYSICAL FUNCTION AND DETECTION SYSTEM

(71) Applicant: Sivantos Pte. Ltd., Singapore (SG)

(72) Inventor: Julius Krieg, Erlangen (DE)

(21) Appl. No.: 17/979,146

(22) Filed: Nov. 2, 2022

(30) Foreign Application Priority Data
Nov. 2, 2021 (DE) 10 2021 212 331.7

(51) Int. Cl. H04R 25/00

(52) U.S. Cl. CPC H04R 25/652; H04R 25/552; H04R 25/60

(57) ABSTRACT

A method for testing a human physical function uses a system wearable on the head of a wearer. A first sensor of the system, which is wearable on a lateral first side of the head, carries out a measurement of a first parameter, which gives information about sweat secreted on the first side of the head. A second sensor of the system, which is wearable on a second side of the head opposite to the first side, carries out a measurement of a second parameter, which gives information about sweat secreted on the second side of the head. A final parameter is ascertained for unilateral anhidrosis of a wearer of the system on the basis of the first parameter and the second parameter. An arrangement for testing a human physical function and a system wearable on the head of a wearer are also provided.

### CROSS-REFERENCE TO RELATED APPLICATION

This application claims the priority, under 35 U.S.C. § 119, of German Patent Application DE 10 2021 212 331.7, filed Nov. 2, 2021; the prior application is herewith incorporated by reference in its entirety.

### FIELD AND BACKGROUND OF THE INVENTION

The invention relates to a method and arrangement for testing a human physical function, wherein a measurement of a first parameter is carried out by a first sensor of a system fastenable on the body at a first point of a body, a measurement of a second parameter is carried out by a second sensor of the system at a second point of the body, and a final parameter for a physical function is ascertained on the basis of the first parameter and the second parameter. The invention also relates to a detection system.

So-called Horner syndrome is a particular type of damage of nerves of the sympathetic trunk in the region of the head and the neck. The damage which results hi Horner syndrome usually only affects one side of the nerve pathways and ganglia. The symptoms of Horner syndrome usually include, due to an impairment of the upper neck ganglion responsible for an eye, a pupil constriction (miosis) drooping of the upper lid (ptosis), and an (apparent) retraction of the eye into the eye socket (pseudo-enophthalmos), and usually occur unilaterally. In addition, the movement of the eyelid and/or the pupils are often generally inhibited (for example in the case of absent ciliospinal reflex, thus the absence of ipsilateral dilation of the pupil after a strong pain stimulation in the facial, shoulder, or neck region).

Detecting Horner syndrome is important in particular because it often represents a symptom of another underlying illness. These include severing of a common carotid artery, an internal carotid artery, or a vertebral artery, Wallenberg syndrome (an infarction in which a spinal artery or a posterior inferior cerebral artery is blocked), or also injuries in the region of the cervical or thoracic spinal column. Moreover, tumor diseases such as thyroid cancer, neuroblastoma, or a so-called Pancoast tumor can induce a Horner syndrome.

While the three first-mentioned, usually life-threatening injuries require immediate medical care, in the case of the tumors the hazard is more not to detect them in a timely manner. This is true in particular for the Pancoast tumor, a rapidly spreading bronchial carcinoma which then also spreads to other organs, and which usually progresses without lung-typical symptoms such as severe coughing or the like, however, and is therefore particularly difficult to detect in the early stage—before spreading to other organs. A reliable detection of a Horner syndrome is therefore of great importance in this case in particular.

In addition to the above-mentioned impairments of the eye, which usually occur unilaterally, unilateral weakening or absence of the sweat secretion often also occurs in Horner syndrome, so-called unilateral anhidrosis. A typical examination for Horner syndrome usually includes the four criteria miosis, ptosis, (pseudo-) enophthalmos (see above), and unilateral anhidrosis. Of the mentioned criteria, unilateral anhidrosis is usually the most difficult to establish for an attending physician.

### SUMMARY OF THE INVENTION

It is accordingly an object of the invention to provide a method and an arrangement for testing a human physical function and a detection system, which overcome the hereinafore-mentioned disadvantages of the heretofore-known methods, apparatuses and systems of this general type and which are capable of assisting a detection of unilateral anhidrosis and making the detection of unilateral anhidrosis particularly easy.

With the foregoing and other objects in view there is provided, in accordance with the invention, a method for testing a human physical function, wherein a measurement of a first parameter is carried out by a first sensor of a system wearable on the head on a lateral first side of a head, which gives information about sweat secreted on the first side of the head, a measurement of a second parameter is carried out by a second sensor of the system on a second side of the head opposite to the first side, which gives information about sweat secreted on the second side of the head, and a final parameter for unilateral anhidrosis of a wearer of the system is ascertained on the basis of the first parameter and the second parameter. Advantageous embodiments, which are partially inventive as such, are the subject matter of the dependent claims and the following description.

With the objects of the invention in view, there is also provided an arrangement for testing a human physical function, comprising a system wearable on the head and having a first sensor, a second sensor and a control unit, the first sensor is disposed, in the intended operation of the system, on a lateral first side of a head of a wearer, and the second sensor is disposed on a second side opposite to the first side, the first sensor and the second sensor are configured to respectively measure a first parameter or second parameter, which gives information about sweat secreted on the first side or second side of the head, and the control unit is configured to ascertain a final parameter for unilateral anhidrosis of a wearer of the system on the basis of the first parameter and the second parameter.

The arrangement according to the invention shares the advantages of the method according to the invention. The advantages indicated for the method and its refinements can be transmitted analogously to the arrangement.

In this case, a system wearable on the head includes in particular a binaural hearing system having a first and a second hearing instrument, which are to be worn by the wearer respectively on one and on the other ear, wherein the first sensor is disposed in the first hearing instrument and the second sensor is disposed in the second hearing instrument. Such a binaural hearing system having two hearing aids usually already intrinsically has the first and the second sensor for measuring the two parameters.

The hearing instruments can be provided in particular in this case by hearing aids in the narrower meaning, which are provided and configured to treat a hearing loss of the wearer.

However, the hearing instruments can also be provided by headphones, which are primarily provided for the playback of audio content (for example, music or speech files), but also for telecommunication (by using corresponding microphones in the hearing instruments). However, in particular also glasses equipped with corresponding sensors, preferably so-called data glasses (for example, “Google glass”) can also be provided as a system wearable on the head.

The first sensor is preferably disposed in this case so that it carries out a measurement in the region of the ear or behind the ear on its relevant side of the head. This applies accordingly to the second sensor on the other side of the head.

A measurement of a parameter which gives information about sweat secreted on the corresponding side of the head is to be understood in particular to mean that a physical variable is ascertained by the relevant sensor, which has a quantitative relationship to the sweat secretion of the skin in the region of the sensor, thus respectively an electrical resistance or impedance of the skin (i.e., an electrodermal activity), a skin reflectance, or also a humidity in the ear. The parameter can then be provided either by the physical variable itself or a variable derived therefrom for the sweat secretion, which is possibly scaled accordingly for the purpose.

The final parameter can have a continuous value range in this case, for example, as a probability value, or as a possibly scaled difference or correlation of the first and second parameter (or as a continuously monotonous function of the difference or correlation). With increasing difference or decreasing correlation, the probability of unilateral anhidrosis thus increases.

However, the final parameter can also be provided as a binary value, in that, for example, for the formation of the final parameter, a difference or correlation of the first and second parameters is compared to a corresponding limiting value, and if an upper limiting value, which is to be selected suitably, for the difference is exceeded or a lower limiting value for the correlation is not reached, sufficiently certain existence of unilateral anhidrosis can be concluded. This can be carried out, for example, on the basis of a quotient of the first and the second parameter at a given point in time.

Electronic devices which are worn by a wearer directly on the body have increasingly complex sensors, through the use of which the increasing monitoring of specific physical functions is enabled during the operation of such a device. In particular devices to be worn on the head, for example, hearing instruments and in this case in particular hearing aids, which are provided and configured to treat a hearing loss of the wearer, but also data glasses or special headset headbands, have increasingly more sensors, through the use of which individual physical functions may be monitored.

Through the use of such a system wearable on the head, which can thus include two hearing instruments or data glasses or a headset headband, upon appropriate equipment of the system with the first and second sensors at suitable points, in particular a repeated or also running measurement of the physical parameters is possible, which permit an inference of the sweat secretion on the left and the right sides of the head and/or neck, and thus underlie the first and the second parameter.

The mentioned devices often already have suitable sensors for the measurement for other purposes, so that an additional reconfiguration of the structure is not even required to carry out the method.

The method now enables already existing infrastructure in the mentioned device types to be used for the repeated or running measurement in particular of the first and the second parameter, so that a very reliable statement can be made as to whether unilateral anhidrosis is present. Alternatively or also additionally thereto, a very informative final parameter can be provided to a physician—for example, in the form of a probability value—for unilateral anhidrosis.

Such a statement or informative final parameter can moreover be created without the action of a physician or a caregiver, so that the statement or final parameter can already be provided upon an examination by a physician or by a caregiver.

The measurement of the first or the second parameter is preferably carried out in each case as a measurement of the electrodermal activity, thus in particular an electrical resistance or impedance of the skin, and/or as a measurement of the skin reflectance on the first or second side of the head. An electrical conductivity and a reflectance (a degree of reflection for incident light) are increased at the relevant point by a sweat secretion.

For the case that a binaural hearing system having a first and a second hearing instrument is used, it is advantageous that a degree of a fitting strength is ascertained in each case for the first hearing instrument and/or the second hearing instrument, wherein the first parameter or the second parameter is additionally ascertained on the basis of the associated degree of the fitting strength, and wherein the degree of the fitting strength is respectively ascertained for each of the two hearing instruments on the basis of a capacitive sensor and/or a photoplethysmographic sensor and/or a pressure sensor and/or at least one transfer function, which represents a propagation of a sound signal from a loudspeaker of the respective hearing instrument to a microphone of the same hearing instrument, or which represents a difference between one microphone of the respective hearing instrument and a further microphone of the same hearing instrument (so-called relative transfer function of one microphone to the other microphone), and/or binary transfer functions, which represent a propagation of a sound signal from a loudspeaker of the respective hearing instrument to two different microphones of the same hearing instrument. In the last-mentioned case of two microphones (and a relative transfer function of the two microphones or the two respective transfer functions for the sound propagation from one loudspeaker), these are preferably spatially separated from one another.

If a reduced degree of the fitting strength is ascertained in this case for one of the two hearing instruments, so that the relevant hearing instrument is thus not optimally seated at its intended wearing point on the auditory canal, the contact of the associated sensor with the adjoining skin areas is thus also reduced. The relevant first or second parameter can then be scaled accordingly in accordance with the ascertained degree of the fitting strength. The degree of the fitting strength is preferably to be ascertained in a physically similar manner as the respective parameter (thus, for example, a capacitive measurement of the fitting strength during a measurement of the electrodermal activity for the parameter). The scaling of the respective measured variable to ascertain the first or second parameter does not necessarily take place in this case in linear dependence on the degree of the fitting strength. In particular, the respective degree of the fitting strength can also be used for the purpose of ascertaining a previously determined, tabulated value for the scaling.

In the mentioned case of the binaural hearing system, in an advantageous alternative embodiment, or also additionally to the above-mentioned measuring method, the measurement of the first or the second parameter is carried out in each case as a measurement of a humidity in the relevant auditory canal and/or as a measurement of a cerumen contamination of the respective hearing instrument. Hearing instruments, and in this case in particular hearing aids in the stricter meaning, often have a sensor for measuring a humidity in the auditory canal. This can be the case, for example, for an active venting system. A cerumen contamination can also be ascertained in this case in a charger of the hearing instruments. In this case, the respective sensor of the system wearable on the head is solely assigned to the system, and is not directly disposed in the system, or the charger having the corresponding sensors is to be viewed as a part of the system, wherein only the hearing instruments of the system are wearable on the head.

The first parameter and the second parameter are advantageously transmitted from the system to an external device, wherein the final parameter for the unilateral anhidrosis of the wearer of the system is ascertained by using the external device. In this way, computing operations to ascertain the final parameter and possibly the storage of values of the two parameters over a longer time period can be outsourced to the external device. The external device can be provided in particular in this case by a smart phone, a smartwatch, a fitness wristband, or a comparable electronic device, which a user wears on the body or carries along for the intended operation. In one embodiment of the arrangement, the control unit is thus disposed in this case in such an external device, which is connectable by a datalink (for example, by Bluetooth or the like) to the system wearable on the head.

The final parameter for the unilateral anhidrosis is preferably ascertained on the basis of a correlation measure, which is determined on the basis of a value of the first parameter and a value of the second parameter.

In particular, the measurement of the first and the second parameter can be carried out at a plurality of points in time, and the final parameter for the unilateral anhidrosis can be ascertained on the basis of the values of the first and the second parameter at the points in time.

This is preferably carried out in that for at least some of the points in time a correlation measure, for example, a correlation coefficient and/or a random sample variance, of the relevant values of the first parameter and the second parameter is ascertained, and the final parameter for the unilateral anhidrosis is ascertained on the basis of the correlation measure ascertained for the points in time. For example, for the points in time, the physical variable which underlies the first and the second parameter is measured in each case on the left and the right side of the head or the neck. These values can now be individually compared to one another for each point in time, or a correlation measure, for example, a random sample variance, can be formed for the two time series, which each result from the values which are measured by the first or the second sensor. In particular a relative deviation can also be used in this case for the correlation measure.

In one advantageous embodiment, the final parameter for the unilateral anhidrosis is ascertained in this case as a probability value from the correlation measure ascertained for the points in time, or as a binary value in dependence on the correlation measure ascertained for the points in time not reaching a lower limiting value. Not reaching the lower limiting value as a criterion can be defined in this case as a single event for a single one of the points in time or a specific minimum number of the points in time (for example, not reaching it for at least three successive points in time or five points in time within a predetermined time period, for example, an hour or a day), or for a time average over a predetermined time (for example, for an hour or a day or a week).

A notification is expediently output to the wearer and/or to a caregiver of the wearer and/or to a medical service if the final parameter for the unilateral anhidrosis assumes a critical value. A medical service is to include for this purpose in particular a resident physician and a hospital or the like, but an automated service can also be provided (for example, by a server of a public care or health system), which passes on the corresponding notification to a physician or caregiver. The notification to the wearer can take place in this case in the case of a binaural hearing system in particular by an automatic speech output by loudspeakers of the hearing instruments.

In one embodiment of the arrangement, the first sensor and/or the second sensor are each provided in this case by a PPG sensor and/or a capacitive sensor and/or an electromyography sensor (EMG) and/or an electrocardiography sensor (ECG), and/or an electroencephalographic sensor (EEG) and/or a humidity sensor and/or a skin conductivity sensor (electrodermal activity, “EDA” or galvanic skin response, “GSR”) and/or a reflectometry sensor and/or a cerumen sensor and/or an arrangement which has a light source and a photodetector. An inference about a sweat secretion can be carried out in this case in particular by measuring, ascertaining, and/or deriving a skin resistance and/or a reflected amount of light from the respective measurement data.

Other features which are considered as characteristic for the invention are set forth in the appended claims.

Although the invention is illustrated and described herein as embodied in a method and an arrangement for testing a human physical function and a detection system, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a block diagram of an arrangement made up of a binaural hearing system having two hearing aids and a smart phone, which is configured for testing a wearer of the hearing system for unilateral anhidrosis;

FIG. 2 is a block diagram of a method for testing a wearer of the hearing system according to FIG. 1 for unilateral anhidrosis; and

FIG. 3 is a block diagram similar to FIG. 1 of an arrangement including a binaural hearing system having two hearing aids and a smart phone, wherein the hearing aids are integrated in a pair of data glasses.

### DETAILED DESCRIPTION OF THE INVENTION

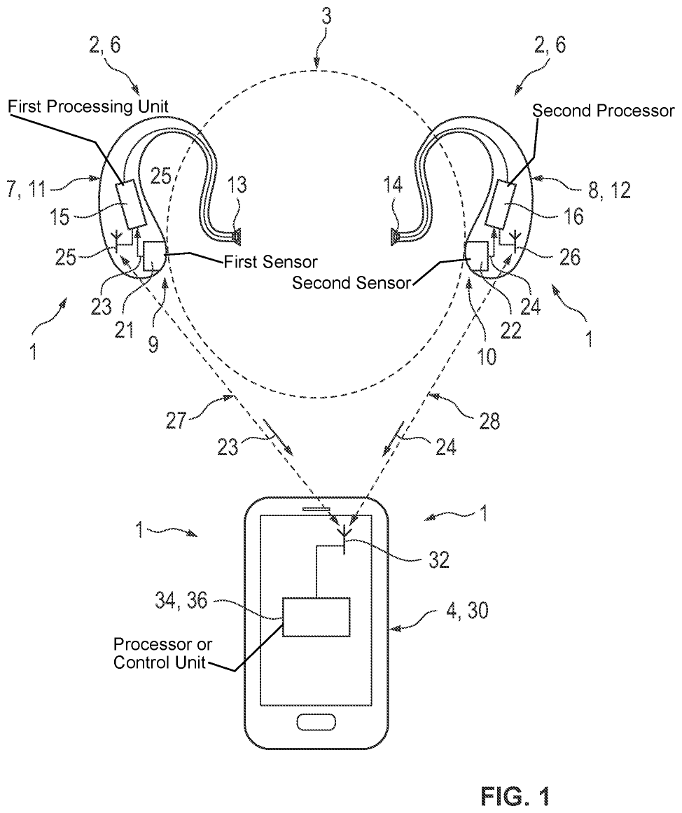

Referring now in detail to the figures of the drawings, in which parts and variables corresponding to one another are each provided with the same reference signs, and first, particularly, to FIG. 1 thereof, there is seen an expanded block diagram of an arrangement 1 which includes a (wearable) system 2 to be worn by a user (not shown in greater detail) on his or her head 3 and an external device 4 connectable by a datalink to the system 2 wearable on the head 3. The head 3 (dashed lines) is solely indicated for this purpose in FIG. 1. The system 2 wearable on the head 3 is provided in the present case by a binaural hearing system 6 having a first hearing instrument 7 and a second hearing instrument 8. The first and the second hearing instrument 7, 8 are respectively provided in the present case by a first hearing aid 11 and a second hearing aid 12, which are to be worn respectively on the left or right ear in the intended operation of the binaural hearing system 6 by the user, thus the wearer of the binaural hearing system 6 (according to the assignment, the first hearing aid 11 can either be worn in this case on the left or on the right ear). The first hearing instrument 7 and the second hearing instrument 8 could just as well also be provided by headphones of a headphone system or a headset.

The first hearing aid 11 has a first loudspeaker 13, which is connected to a first processor unit 15 of the first hearing aid 11 and converts an output signal output thereby into a corresponding output sound. Similarly thereto, the second hearing aid 12 has a second loudspeaker 14, which is connected to a second processor unit 16 of the second hearing aid 12 and also converts an output signal output thereby into a corresponding output sound. In addition, the first hearing aid 11 and the second hearing aid 12 have still further components, which are solely indicated in FIG. 1 and which are provided in the respective hearing aid 11, 12 to treat a hearing loss of the wearer of the binaural hearing system 6, so that the treatment can be carried out by the first and the second hearing aid 11, 12 (and these are configured accordingly).

The first hearing aid 11 has a first sensor 21, which is connected to the first processing unit 15, and which is configured when worn on the ear as intended to measure a first parameter 23 on a lateral first side 9 of the head 3, which gives information about a sweat secretion of the wearer at the relevant measurement point of the first side 9. This can be carried out, for example, by an optical measurement of the skin reflectance or by a measurement of the electrodermal activity, thus a measurement of the skin conductivity (or of the electrical resistance of the skin).

Similarly thereto, the second hearing aid 12 has a second sensor 22, which is connected to the second processor unit 16 and which is configured, wherein the second hearing aid 12 is worn as intended on a second side 10 of the head 3 opposite to the first side 9, to measure a second parameter 24 which gives information about a sweat secretion of the wearer at the second measurement point of the second side 10 (thus, for example, with a BTE hearing aid region behind the ear). The first sensor 21 and the second sensor 22 are preferably configured identically (and thus use the same physical measuring principal in both cases). The system 2 wearable on the head could also be provided in an alternative (not shown) by data glasses, which are equipped with the first and the second sensor 21, 22.

In an alternative embodiment of the invention, still further sensors than the first and the second sensor 21, 22 can also be used for the measurement of the first and the second parameter 23, 24 in this case.

The first parameter 23, which was measured by the first sensor 21, is now converted in the first processor unit 15 into a transmittable data format, and transmitted over a first connection 27, which is established by using a first antenna 25 of the first hearing aid 11 connected to the first processor unit 15, to the external device 4. In a comparable manner, the second parameter 24 is also transmitted over a second connection 28, which is established by using a second antenna 26 of the second hearing aid 12 connected to the second processor unit 16, to the external device 4.

The external device 4 is provided in the present case by a smart phone 30 having an antenna 32 and a control unit 36 configured as a processor unit 34, but could also be provided as a smartwatch or comparable device, which is in particular carried along by the user or worn on the body for the intended operation. The processor unit 34 has in this case at least one processor core and an operating memory addressable thereby and possibly also a nonvolatile memory for storing an operating program and/or operating system.

The first and the second antenna 25, 26 and the antenna 32 of the smart phone 30 stand generically in this case for corresponding communication directions, which are suitable and configured to establish the first and the second connections 27, 28 between the first or second hearing aids 11, 12, respectively, and the smart phone 30.

In the control unit 36, the first and the second parameters 23, 24 are assessed in a way still to be described in order to output a final parameter with respect to unilateral anhidrosis of the wearer of the binaural hearing system 6. The ascertainment of the final parameter can take place in this case in an alternative embodiment of the invention also in the binaural hearing system 6 itself, for example, in the first and/or second processor unit 15, 16 as an alternative embodiment (not shown) to the control unit (36), or also in a charging station, which is provided with corresponding computing capacity and configured for this purpose, for the first and the second hearing aid 11, 12.

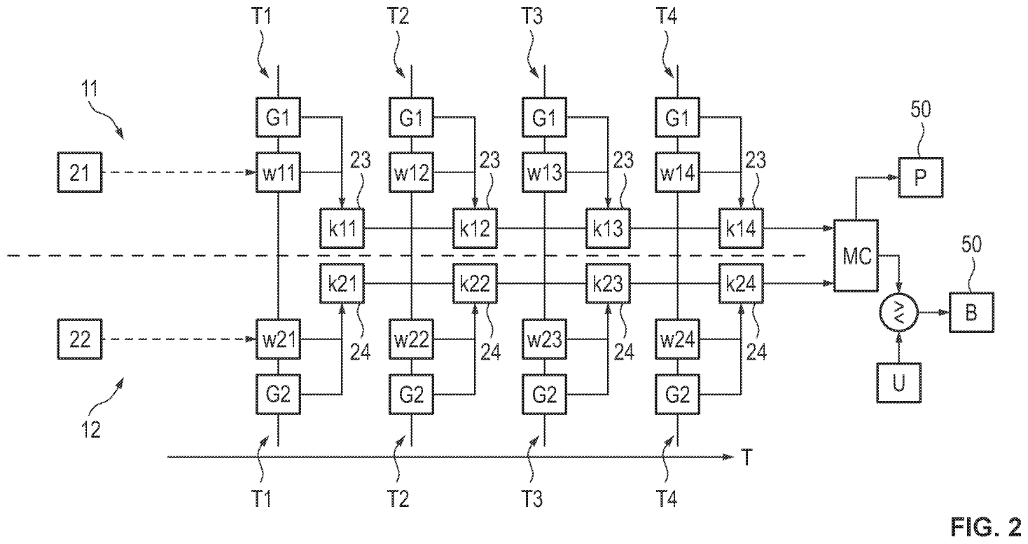

In FIG. 2, a course of a method, with which a final parameter for unilateral anhidrosis can be established for the wearer of the binaural hearing system 6 according to FIG. 1, is schematically shown in a block diagram against a time axis T.

At a plurality of points in time T1, T2, T3, T4, in the embodiment of the invention shown in FIG. 2, measured values w11, w12, w13, w14 as a precursor for the first parameter 23 and measured values w21, w22, w23, w24 as a precursor for the second parameter 24 are measured by the first sensor 21 and the second sensor 22 of the first or second hearing aid 11, 12, respectively. The measured values w11, . . . , w24 can be provided in this case, for example, by specific values of a skin reflectance or a conductivity of the skin (depending on the physical embodiment of the first and the second sensor 11, 12).

In addition, a degree G1, G2 of a fitting strength is ascertained continuously for each of the two hearing aids 11, 12. This can preferably take place at each of the indicated points in time T1, T2, T3, T4, thus G1(T1), G2(T1), G1 (T2), etc. The respective degree G1, G2 of the fitting strength can be ascertained in each case, for example, on the basis of a capacitive sensor (not shown), which determines the strength of a contact of the respective hearing aid 11, 12 with the skin in the region of the associated first or second sensor 21, 22, respectively, or also through a PPG sensor for which such a strength of the contact may be estimated on the basis of the signal strength.

On the basis of the respective degree G1 of the fitting strength of the first hearing aid 11, the individual measured values w11-w14 are now weighted accordingly, thus multiplied by a corresponding scaling factor, which is ascertained in each case from the degree G1 of the fitting strength (for example, on the basis of previously tabulated values which are determined by calibration measurements), and thus respective values k11, k12, k13, and k14 for the first parameter 23 are ascertained for the plurality of points in time T1, T2, T3, and T4.

In a comparable manner, on the basis of the respective degree G2 of the fitting strength of the second hearing aid 12, the individual measured values w21-w24 are scaled, and thus corresponding values k21, k22, k23, and k24 for the second parameter 24 are ascertained.

In the embodiment of the invention illustrated in FIG. 1, the values k11, . . . , k14 of the first parameter 23 and the values k21, . . . , k24 for the second parameter 24 are formed directly by the measured values w11, . . . , w14 or w21, . . . , w24 measured by the first or second sensor 21, 22, respectively.

A correlation measure MC is now ascertained from the values k11, k12, k13 and k14 for the first parameter 23 at the points in time T1, T2, T3, and T4 and the corresponding values k21, k22, k23, and k24 for the second parameter 24. The correlation measure can be formed in this case, for example, by a correlation coefficient or a random sample covariance of the mentioned values for the two parameters 23, 24. There is also a possibility of suitably weighted averaging over multiple correlation variables to generate the correlation measure MC.

If the values k11, . . . , k14 for the first parameter 23 are strongly correlated in this case with the values k21, . . . , k24 for the second parameter, this thus means that the sweat secretion is strongly correlated on both sides at the plurality of points in time T1, . . . , T4 and therefore there is not a high probability for unilateral anhidrosis. However, if those values have a low correlation, this thus indicates that the sweat secretion on one side is suppressed or even substantially suppressed, and is thus reduced or even largely does not occur independently of a physical exertion or a rest phase of the wearer.

A probability value P for a presence of unilateral anhidrosis in the wearer of the binaural hearing system 6 is now therefore ascertained from the correlation measure MC in that the correlation measure MC is mapped in a suitable manner on the interval [0, 1]. If this also applies to the correlation measure MC∈[0, 1], for example, an anti-linear mapping of the form P (MC)=1−MC can thus also be used; however, monotonous scaling can be carried out in this case, for example, in the form P (MC)=1−x² or the like.

The probability value P can now be used as the final parameter 50 for a presence of unilateral anhidrosis. Alternatively thereto, such a final parameter 50 for a presence of unilateral anhidrosis can also be ascertained as a binary value B from the correlation measure MC. The binary value B assumes the value 0 if the correlation measure MC is above a lower limiting value U (and thus sufficiently high correlation of the values k11, . . . , k24 for the parameters 23, 24 is present on both sides), and the value 1 if the correlation measure MC is below the lower limiting value U, thus U<MC (and therefore the sweat secretion on both sides of the head is uncorrelated, so that a malfunction on one side can be presumed).

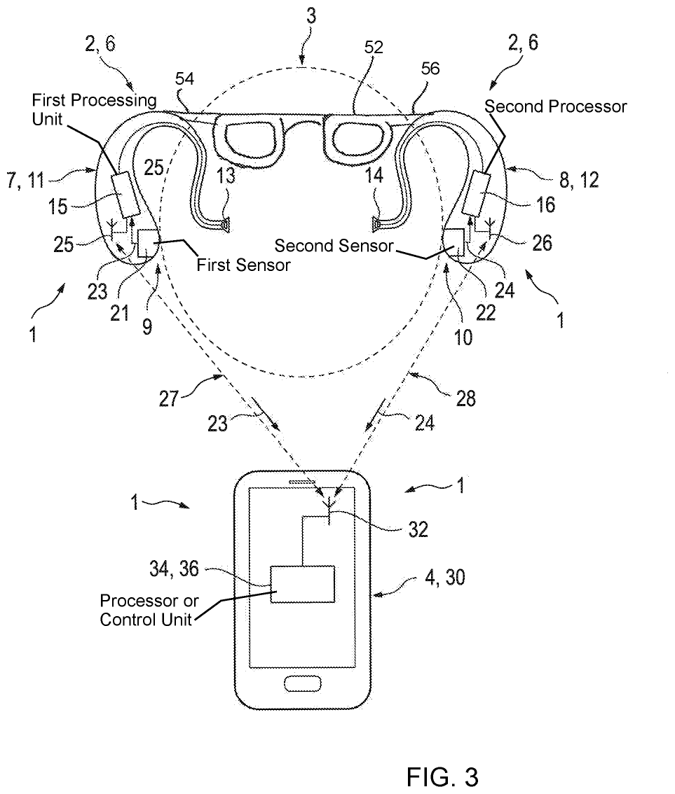

In the embodiment of FIG. 3, the system 2 wearable on the head 3 is formed by a pair of (data) glasses 52 in which a binaural hearing system 6 according to FIG. 1 is integrated. In particular, the first hearing instrument 7 of the hearing system 6 is integrated in a first temple 54 of the glasses 52, and the second hearing instrument 8 of the hearing system 6 is integrated in a second temple 56 of the glasses 52.

Although the invention was illustrated and described in more detail by the preferred exemplary embodiment, the invention is not thus restricted by the disclosed examples; other variations can be derived therefrom by a person skilled in the art without leaving the scope of protection of the invention.

The following is a summary list of reference numerals and the corresponding structure used in the above description of the invention.

### LIST OF REFERENCE SIGNS

1 arrangement
2 system wearable on the head
4 external device
6 binaural hearing system
7 first hearing instrument
8 second hearing instrument
9 first side
10 second side
11 first hearing aid
12 second hearing aid
13 first loudspeaker
14 second loudspeaker
15 first processor unit
16 second processor unit
21 first sensor
22 second sensor
23 first parameter
24 second parameter
25 first antenna
26 second antenna
27 first connection
28 second connection
30 smart phone
32 antenna
34 processor unit
36 control unit
50 final parameter
52 (data) glasses
54 temple
56 temple
B binary value
G1 degree of a fitting strength of the first hearing aid
G2 degree of a fitting strength of the second hearing aid
k11 . . . k14 value (for the first parameter)
k21 . . . k24 value (for the second parameter)
MC correlation measure
P probability value
T time axis
T1 . . . T4 point in time
U lower limiting value
w11 . . . w14 measured value (of the first sensor)
w21 . . . w24 measured value (of the second sensor)

1. A method for testing a human physical function, the method comprising:
providing a system wearable on the head of a wearer;
using a first sensor of the system configured to be disposed on a lateral first side of the head to carry out a measurement of a first parameter giving information about sweat secreted on the first side of the head;
using a second sensor of the system configured to be disposed on a side of the head opposite to the first side to carry out a measurement of a second parameter giving information about sweat secreted on the second side of the head; and
ascertaining a final parameter for unilateral anhidrosis of the wearer of the system based on the first parameter and the second parameter.

2. The method according to claim 1, which further comprises carrying out the measurement of each respective first or second parameter as at least one of a measurement of electrodermal activity or a measurement of skin reflectance on the respective first or second side of the head.

3. The method according to claim 1, which further comprises:
providing a binaural hearing system having a first hearing instrument and a second hearing instrument as the system wearable on the head;
providing the first sensor in the first hearing instrument and providing the second sensor in the second hearing instrument.

4. The method according to claim 3, which further comprises:
ascertaining a degree of a fitting strength for at least one of the first hearing instrument or the second hearing instrument;
ascertaining the first parameter or the second parameter based on the respective degree of the fitting strength; and
ascertaining the degree of the fitting strength for each respective hearing instrument based on at least one of:
a capacitive sensor, or
a photoplethysmographic sensor, or
a pressure sensor, or
at least one transfer function representing a propagation of a sound signal from a loudspeaker of the respective hearing instrument to a microphone of a same hearing instrument, or representing a difference between one microphone of the respective hearing instrument and a further microphone of a same hearing instrument.

5. The method according to claim 3, which further comprises carrying out the measurement of each of the first or second parameter as at least one of a measurement of a humidity in a relevant auditory canal or a measurement of a cerumen contamination of the respective hearing instrument.

6. The method according to claim 1, which further comprises:
providing glasses or data glasses as the system wearable on the head; and
placing each of the first sensor and the second sensor on a respective one of a left side and a right side of the glasses.

7. The method according to claim 1, which further comprises:
transmitting the first parameter and the second parameter from the system to an external device; and
ascertaining the final parameter for the unilateral anhidrosis of the wearer of the system by using the external device.

8. The method according to claim 1, which further comprises ascertaining the final parameter for the unilateral anhidrosis based on a correlation measure determined on a basis of a value of the first parameter and a value of the second parameter.

9. The method according to claim 8, which further comprises:
carrying out the measurement of the first and second parameters at a plurality of points in time; and
ascertaining the final parameter for the unilateral anhidrosis based on the values of the first and second parameters at the points in time.

10. The method according to claim 9, which further comprises:
ascertaining a correlation measure of the relevant values of the first parameter and the second parameter for at least some of the points in time; and
ascertaining the final parameter for the unilateral anhidrosis based on the correlation measure ascertained for the points in time.

11. The method according to claim 10, which further comprises:
ascertaining the final parameter for the unilateral anhidrosis as a probability value from the correlation measure ascertained for the points in time, or
ascertaining the final parameter for the unilateral anhidrosis as a binary value in dependence on the correlation measure ascertained for the points in time not reaching a lower limiting value.

12. The method according to claim 1, which further comprises outputting a notification to at least one of the wearer or a caregiver of the wearer or a medical service when the final parameter for the unilateral anhidrosis assumes a critical value.

13. An arrangement for testing a human physical function, the arrangement comprising:
a system wearable on the head of a wearer, said system including a first sensor, a second sensor, and a control unit;
said first sensor configured to be disposed on a lateral first side of the head of the wearer, and said second sensor configured to be disposed on a lateral second side of the head of the wearer opposite to the first side, during an intended operation of said system;
said first sensor and said second sensor each configured to measure a respective first parameter or second parameter giving information about sweat secreted on the first side or the second side of the head; and
said control unit configured to ascertain a final parameter for unilateral anhidrosis of the wearer of said system based on said first parameter and said second parameter.

14. The arrangement according to claim 13, wherein:
said system is a binaural hearing system having a first hearing instrument and a second hearing instrument; and
said first sensor is disposed in said first hearing instrument and said second sensor is disposed in said second hearing instrument.

15. The arrangement according to claim 13, which further comprises an external device configured to be connected by a data link to said system wearable on the head, said control unit being disposed in said external device.

16. The arrangement according to claim 13, wherein at least one of said first sensor or said second sensor is at least one of:
a photoplethysmographic sensor, or
a capacitive sensor, or
an electromyography sensor, or
an electrocardiography sensor, or
an electroencephalography sensor, or
a humidity sensor, or
a skin conductivity sensor, or
a reflectometry sensor, or
a cerumen sensor, or
an arrangement having a light source and a photodetector.

17. A system wearable on the head of a wearer, the system comprising:
a first sensor and a second sensor for detecting unilateral anhidrosis of a wearer of the system;
said first sensor configured to be disposed on a lateral first side of the head of the wearer, and said second sensor configured to be disposed on a lateral second side of the head of the wearer opposite to the first side, during an intended operation of the system;
said first sensor and said second sensor each configured to measure a respective first parameter or second parameter giving information about sweat secreted on the first side or the second side of the head; and
said first parameter and said second parameter providing a basis for ascertaining a final parameter for unilateral anhidrosis of the wearer.
